(19) United States
(12) Patent Application Publication
(10) Pub. No.: US 2009/0004132 A1
(43) Pub. Date: Jan. 1, 2009

(54) Transfer resistant cosmetic composition with improved feel

(75) Inventors: John Richard Nicholson, Ramsey, NJ (US); Lara Priscila Fieschi-Corso, Danbury, CT (US)

Correspondence Address: DILWORTH & BARRESE, LLP, 333 EARLE OVINGTON BLVD., SUITE 702, UNIONDALE, NY, 11553, US

(73) Assignee: Momentive Performance Materials Inc.

(21) Appl. No.: 11/821,801

(22) Filed: Jun. 26, 2007

(51) Int. Cl. A61K 8/92; A61K 8/72; A61Q 1/04; A61Q 1/06; A61Q 1/08; A61Q 1/10; A61Q 1/12

(52) U.S. Cl. 424/78.03; 424/64; 424/69; 424/70.7

(57) ABSTRACT

A transfer resistant cosmetic composition with improved feel is provided which comprises:
a) a film-forming effective amount of at least one silicone-aliphatic hydrocarbon wax in which the aliphatic hydrocarbon substituent possesses from 24 to 60 carbon atoms;
b) a transfer resistant effective amount of at least one silicone resin selected from the group consisting of MQ resin and MT resin; and,
c) at least one volatile carrier,
the cosmetic composition being substantially free of dimethicone gum.

### BACKGROUND OF THE INVENTION

(1) Field of the Invention

This invention relates to cosmetic compositions and, in particular, to transfer resistant cosmetic compositions for application to the skin.

(2) Description of Related Art

A need exists for cosmetic compositions that provide long wear benefits while also feeling natural, or comfortable, when applied to the skin. A number of cosmetic compositions have been described in the prior art that are said to be highly substantive to the skin and provide long-wear. However, the film-forming components(s) that are typically utilized in such compositions due to their relatively limited flexibility eventually result in cracking or peeling of the composition, a phenomenon that is uncomfortable to the wearer and often unsightly as well.

Polymers are often incorporated into cosmetic compositions to form a film that will impart long-wear benefits. These include natural polymers such as cellulosics, gums and resins and also synthetic polymers. Synthetic polymers typically fall into two classes: silicone polymers and organic polymers. Organic polymers are formed through polymerization of ethylenically unsaturated monomers such as acrylates or alkylenes. While synthetic polymers comprised of organic moieties are excellent film-formers, they do not always exhibit the degree of flexibility or plasticity that is desired for application to a skin surface that is in constant movement. Silicone polymers can be excellent film-formers and provide strong adhesion and excellent wear. However, they are not always compatible with other cosmetic ingredients used in the formulation of cosmetic products having desirable properties.

The use of silicone resins for providing benefits of long wear in cosmetic compositions, e.g., lipsticks, lip glosses, etc., is known. A frequent drawback of these compositions is that they do not provide a high level of comfort when applied to the skin.

### SUMMARY OF THE INVENTION

In accordance with the present invention, a transfer resistant cosmetic composition with improved feel is provided which comprises:
a) a film-forming effective amount of at least one silicone-aliphatic hydrocarbon wax in which the aliphatic hydrocarbon substituent possesses from 24 to 60 carbon atoms;
b) a transfer resistant effective amount of at least one silicone resin selected from the group consisting of MQ resin and MT resin; and,
c) at least one volatile carrier,

the cosmetic composition being substantially free of dimethicone gum.

In addition to providing good resistance to transfer, the foregoing composition exhibits improved comfort to the wearer.

As used herein the term “cosmetic composition” includes makeup, foundation and other skin care products. The term makeup” refers to products that leave color on the face including foundation, blacks and browns, i.e., mascaras, concealers, eye liners, brow colors, eye shadows, blushers, lip colors, powders, solid emulsion compacts, and the like. “Skin care products” are those used to treat or care for, or somehow moisturize, improve, or clean the skin. Products contemplated by the phrase “skin care products” include, but are not limited to, adhesives, bandages, toothpaste, anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams and the like. The term “foundation” refers to liquid, creme, mousse pancake, compact, concealer, or the like, which are intended to even out the overall coloring of the skin. Foundation is manufactured to work better over moisturized and/or oiled skin.

### DETAILED DESCRIPTION OF THE INVENTION

A. Silicone-Aliphatic Hydrocarbon Wax

The silicone-aliphatic hydrocarbon wax component of the cosmetic composition herein provides a thickening of the cosmetic composition and provides critical film-forming properties on the surface of skin to which the composition is applied. Useful silicone-aliphatic hydrocarbon waxes are those in which the aliphatic hydrocarbon substitutuent possesses from 24 to 60, and preferably 30 to 45, carbon atoms. One such silicone wax that can be employed in the cosmetic composition of the invention with generally good results is SF1642 (Momentive Performance Materials Inc.), a C30-45 alkyl dimethicone wax.

The cosmetic composition of the invention will incorporate the silicone wax component at a level sufficient to form a transfer-resistant film when combined with at least one MQ resin and/or at least one MT resin. In general, the amount of silicone wax effective to achieve this can vary from about 3 to about 30, preferably from about 5 to about 20, and more preferably from about 8 to about 15, percent by weight of the cosmetic composition.

B. Silicone Resin

The silicone resin component of the cosmetic composition of the invention provides a lubricious feel to the cosmetic when applied to the skin and upon drying of the cosmetic film, the silicone resin component imparts a significantly enhanced resistance to transfer when combined with the silicone-aliphatic hydrocarbon wax component of the cosmetic composition.

The silicone resin component of the cosmetic composition herein is at least one member selected from the group consisting of MQ silicone resin and MT silicone resin.

As used herein, the term “MQ resin” shall be understood to include both the non-fluorinated and fluorinated MQ resins.

(1) Non-Fluorinated MO Resin

Non-Fluorinated MQ resins (or simply “MQ resins”) are generically known in the art and contain M units, represented by the formula R¹₃SiO_{1/2} and Q units, represented by the formula SiO_{4/2}. It is recognized that while the MQ resins are primarily made from M and Q units, there can be up to 5 mole percent of D units represented by the formula R²₂SiO_{2/2} and T units, represent by the formula R3SiO_{3/2}, wherein in each R^{1,} R² and R³ is independently hydroxyl or a monovalent hydrocarbon radical. Examples of preferred monovalent hydrocarbon radical include those monovalent hydrocarbon radicals having from about 1 to about 6 carbon atoms, such as alkyl radicals including methyl, ethyl, and isopropyl; alkenyl radicals including ethylene, propenyl, and hexenyl; cycloaliphatic radicals such as cyclopentyl cyclohexenyl; olefinic-containing radicals such as vinyl and allyl; and olefinic-containing radicals. Preferably, the monovalent hydrocarbon radical is methyl.

Commercially available non-fluorinated MQ resin which can be used in the cosmetic compositions of the invention include MQ resins such as SR1000, SS4230, and SS4276 available from Momentive Performance Materials and Wacker 803 and 804 available from Wacker Silicones Corporation.

(2) Fluorinated MQ Resin

Fluorinated MQ resins are known in the art, e.g., as described in U.S. Pat. No. 5,777,047, the entire contents of which are incorporated by reference herein. The fluorinated MQ resins contain M units represented by the formula R¹₃SiO_{1/2}, Q units represented by the formula SiO_{4/2}, fluorine-containing groups, preferably bonded to the silicon atoms of M units via oxygen atoms, and, optionally, up to 5 mole percent of D units represented by the formula R²₂SiO_{2/2} and/or T units represented by the formula R³SiO_{3/2}. In the foregoing fluorinated MQ resins, each R¹, R² and R³ is independently a hydroxyl or hydrocarbyl radical. Examples of preferred hydrocarbyl radicals include those possessing from 1 to 6 carbon atoms, e.g., alkyl radicals such as methyl, ethyl and isopropyl; alkenyl radicals such as ethenyl, propenyl and hexenyl; cycloaliphatic radicals such as cyclopentyl and cyclohexyl; and, olefinic-containing radicals such as vinyl and allyl. Preferably, the hydrocarbyl radical is methyl.

Suitable fluorine-containing groups in the fluorinated MQ resins include those of the general formula:

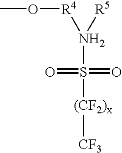

and those of the general formula:

—O—R⁴—(CF₂)ₓCF₃

wherein R⁴ and R⁶ each independently is an alkylene group possessing from 2 to 6 carbon atoms, 0≦x≦12 and each R⁵ independently is an alkyl group possessing from 1 to 6 carbon atoms. In preferred embodiments, the value of x is from 3 to 11 and preferably from 3 to 7 and R⁵ is methyl.

A fluorinated MQ resin which can be used in the cosmetic compositions of the invention with generally good results is XS66-B8226 or XS66-C1191, from Momentive Performance Materials Inc.

The MQ resin component(s) of the cosmetic composition herein, i.e., non-fluorinated MQ resin(s), fluorinated MQ resin(s) or mixture thereof, can be incorporated therein at a level sufficient to improve the non-transfer performance of the cosmetic film when the cosmetic applied to skin. Amounts of fluorinated MQ resin in most cases can vary from about 1 to about 50, preferably from about 2 to about 30 and more preferably from about 5 to about 20, percent by weight of the cosmetic composition.

(3) MT Resin

In silicones of general formula MT, M possesses the formula R₃SiO_{0.5}, e.g., R⁶R⁷RSiO_{0.5}, R⁶R⁷RSiO_{0.5} or R⁶R⁷R⁸SiO_{0.5} wherein R is methyl and R⁶, R⁷ and R⁸ each independently is alkyl of from 1 to 6 carbon atoms, phenyl, diisostearoyl trimethylolpropane or dilauryltrimethylolpropane and T is a trifunctional siloxy unit of the empirical formula R⁹SiO_{1.5} wherein R⁹ is alkyl of up to 20 carbon atoms, optionally substituted with one or more hydroxyl groups and/or halogen atoms.

Preferably, M in the MT resin possesses the formula R₃SiO_{0.5} in which R is methyl and T possesses the formula RSiO_{1.5} in which R is methyl.

MT resins suitable for inclusion in the cosmetic composition of the invention are well known in the art, e.g., polymethylsilsesquioxanes which are especially advantageous for use herein a number of which are commercially available, e.g., Tospearl 145A, Tospearl 3000A, Tospearl 2000B, Tospearl 1110A, Tospearl 150KA, all available from Momentive Performance Materials Inc.

The MT resin is incorporated in the cosmetic composition herein at a level sufficient to provide a soft, smooth and lubricious feel to the cosmetic when first applied to the skin and also upon drying of the cosmetic film, to impart a significantly enhanced resistance to transfer. Amounts of MT resin in most cases can vary from about 1 to about 50, preferably from about 1 to about 30, and more preferably from about 5 to about 20, percent by weight of the cosmetic composition.

When both a fluorinated MQ resin and an MT resin are employed herein, their combined weight can be within the aforestated ranges for the individual resins.

C. Volatile Carrier

In the cosmetic composition of the invention, silicone-aliphatic hydrocarbon wax (a) and silicone resin(s) (b) must be readily transferred to the site of application, e.g., the lips in the case of a lipstick or lip gloss. This transfer requires that aforesaid components (a) and (b) are incorporated in a volatile carrier, one which quickly volatilizes from the site of application leaving a thin, durable, flexible transfer resistant film.

The volatile carrier will ordinarily comprise from about 10 to about 90, preferably from about 15 to about 80, and more preferably from about 20 to about 70, weight percent of the cosmetic composition. The volatile carrier of the present invention is advantageously selected from the group consisting of volatile hydrocarbon oils, volatile silicones and mixtures thereof.

Volatile hydrocarbon oils useful in the present invention include those having boiling points in the range of 60-260° C., preferably isoparaffins possessing from about 8 to about 20 carbon atoms. Of these isoparaffins, preferred are those selected from the group consisting of isododecane, isohexadecane, isoeicosane, 2,2,4-trimethylpentane, 2,3-dimethylhexane and mixtures thereof. More preferred is isododecane such as Permethyl 99A from Permethyl Corporation.

Preferred volatile silicone fluids include cyclomethicones possessing 3, 4 and/or 5 membered ring structures corresponding to the formula:

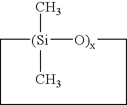

where x is from about 3 to about 6. Such volatile silicones include 244 Fluid, 344 Fluid and 245 Fluid and 345 Fluid, all available from Dow Corning Corporation.

Ranges of amounts of silicone-aliphatic hydrocarbon wax, fluorinated MQ and/or MT silicone resin and volatile carrier for a variety of cosmetic compositions are set forth in Table 1 below:

TABLE 1
Cosmetic Formulations (all amounts by weight percent of the total composition)

| | Silicone-Aliphatic | | | Fluorinated MQ and/or | | | | | |
|---|---|---|---|---|---|---|---|---|---|
| Cosmetic | Hydrocarbon Wax | | | MT Silicone | | | Volatile Carrier | | |
| Composition | Broad | Medium | Narrow | Broad | Medium | Narrow | Broad | Medium | Narrow |
| lipstick | 0.5-50 | 1-30 | 2-20 | 0.01-50 | 0.1-20 | 0.5-15 | 0.1-20 | 1-10 | 2-50 |
| lip gloss | 0.5-40 | 1-30 | 2-20 | 0.01-50 | 0.1-15 | 0.5-15 | 0.1-80 | 2-65 | 3-50 |
| makeup | 0.5-30 | 1-20 | 2-15 | 0.01-50 | 0.1-20 | 0.5-15 | 0.5-70 | 2-50 | 3-50 |
| mascara | 0.5-70 | 1-50 | 2-15 | 0.01-50 | 0.1-30 | 0.5-15 | 0.5-80 | 2-60 | 3-30 |
| eye liner | | | | | | | | | |
| blusher | | | | | | | | | |
| eye shadow | 1-50 | 2-30 | 3-20 | 0.01.50 | 0.1-20 | 0.5-15 | 0.5-70 | 2-50 | 3-20 |

D. Other Components

The cosmetic composition of the invention, depending on its purpose, will ordinarily contain one or more additional components such as colorants such as a pigments, including pearlescent pigments such as, for example, bismuth oxychloride and titanium dioxide coated mica, a water soluble dye or a liposoluble dyes; other waxes; emulsifiers; skin care ingredients; emollients such as, for example, triglyceride esters, wax esters, alkyl or alkenyl esters of fatty acids or polyhydric alcohol esters; humectants; moisturizers; enzymes; medicinal compounds; vitamins such as, for example, Vitamin A, Vitamin C and Vitamin E; salts; electrolytes; alcohols; polyols; absorbing agents for ultraviolet radiation or sunscreen or sunblock compounds, such as, for example, titanium dioxide, zinc oxide, oxybenzone, octylmethoxy cinnamate, butylmethoxy dibenzoyl methane, p-aminobenzoic acid and octyl dimethyl-p-aminobenzoic acid; botanical extracts; surfactants; silicone oils; organic oils; film formers; thickening agents such as, for example, fumed silica or hydrated silica, particulate fillers, such as for example, talc, kaolin, starch, modified starch, mica, nylon, clays, such as, for example, bentonite and organo-modified clays; fragrant materials, which may be encapsulated fragrant compounds, or fragrance releasing compounds that either the neat compounds or are encapsulated etc., in combinations and in amounts that are well known in the art. Similarly, the procedures for combining these and other known and conventional cosmetic ingredients into a finished cosmetic product are also well known to those skilled in the art.

The following examples (Examples 1-4) are illustrative of a cosmetic formulation prepared in accordance with the invention, specifically, a lip color, and are compared with several lip color compositions of conventional formulation (Comparative Examples 1-4) for both color transfer and moisture barrier properties.

(1) Color Transfer Resistance Property

Red-pigmented lip color compositions formulated as shown in Table 2, infra, were applied to Vitro Skin at a loading of 1.4 mg/cm². After allowing 30 minutes for a composition to dry, a white tissue was wiped across the skin surface while applying downward pressure. The intensity of the red color of each lip color composition was measured via L.a.b. color measurements using a HunterLab ColorQuest Spectrophotometer. Specifically, the value of “a” was taken as the measure of color transfer, so that a low value of “a” means that the product is highly resistant to transfer.

TABLE 2
Lip Color Compositions (all amounts in weight percent)

| | Comp. | Comp. | Comp. | Comp. | | | | | |
|---|---|---|---|---|---|---|---|---|---|
| Component | Ex. 1 | Ex. 2 | Ex. 3 | Ex. 4 | Ex. 1 | Ex. 2 | Ex. 3 | Ex. 4 | Ex. 5 |
| High MW dimethicone gum (SE30*) | 10.0 | 10.0 | | 10.0 | | | | | |
| MQ resin blend (75 wt. % SR1000* + 25 wt. % IDD) | | 17.5 | | | | 13.3 | | 5.0 | |
| MT resin (Tospearl 110Au*) | | | | 10.0 | 10.0 | | 10.0 | 10.0 | |
| Fluorinated MQ resin (Fluorosilicate XS66-C1191*) | | | | | | | 5.0 | | 20.0 |
| Alkyl silicone wax (SF1642*) | 2.0 | 2.0 | 12.0 | 2.0 | 12.0 | 12.0 | 12.0 | 12.0 | 12.0 |
| Water-in-oil emulsifier (SF1528*) | 2.6 | 2.6 | 2.6 | 2.6 | 2.6 | 2.6 | 2.6 | 2.6 | 2.6 |
| Isododecane | 53.5 | 36.0 | 53.5 | 43.5 | 43.5 | 40.2 | 38.5 | 38.5 | 38.5 |
| Red Pigment (KOBO C020R7C | 29.0 | 29.0 | 29.0 | 29.0 | 29.0 | 29.0 | 29.0 | 29.0 | 29.0 |
| Titanium Dioxide (100T Tri-K) | 2.9 | 2.9 | 2.9 | 2.9 | 2.9 | 2.9 | 2.9 | 2.9 | 2.9 |

*Components available from Momentive Performance Materials Inc.

Color transfer, as defined by the “a” values measured on white tissue are reported in Table 2 for Comparative Examples 1-4 and Examples 1-4.

In the case of the control composition, Comparative Example 1, which lacks any transfer resistance component, a high level of color transfer is observed (a=20).

TABLE 3
Color Transfer Values (“a” value) measured for
Comparative Examples 1-4 and Examples 1-4

| Example | Color Transfer Value |
|---|---|
| Comp. Ex. 1 | 20 |
| Comp. Ex. 2 | 6 |
| Comp. Ex. 3 | 22 |
| Comp. Ex. 4 | 20 |
| Ex. 1 | 8 |
| Ex. 2 | 5 |
| Ex. 3 | 6 |
| Ex. 4 | 2 |

The composition of Comparative Example 2 contains a high molecular weight dimethicone gum, an MQ resin blend, an alkyl silicone wax and a volatile carrier. This composition exhibits both an acceptably low level of color transfer (a=6) and an acceptably low level of trans-epidermal water loss, an indication that the composition will be perceived by the wearer to be relatively comfortable.

The composition of Comparative Example 3 contains an alkyl silicone wax but no dimethicone gum or silicone resin(s). This composition showed a high level of transfer (a=22), comparable to that of Comparative Example 1, demonstrating that the alkyl silicone wax component of the composition by itself does not impart an adequate transfer resistance benefit when incorporated into a lip color composition.

The composition of Comparative Example 4 contains dimethicone gum, an MT resin and an alkyl silicone wax. However, as seen in Table 2, this composition exhibited a high level of transfer (a=20). Thus, when the organosiloxane resin is an MT resin, such as a polymethylsilsesquioxane, that is combined with a silicone gum, an alkyl silicone wax and a volatile carrier, there is no significant non-transfer benefit provided.

In contrast, the combination of an alkyl silicone wax with a non-fluorinated MQ resin, fluorinated MQ resin and/or MT silicone resin and volatile carrier in accordance with the present invention, but no dimethicone gum (present in the compositions of Comparative Examples 1, 2 and 4), provided very good non-transfer benefits (all color transfer values of 8 or less) as shown by the data for the compositions of Examples 1-4.

In contrast to silicone-aliphatic hydrocarbon waxes, MQ resins and MT resins do not form a film nor are they soluble in, or compatible with, the other components of known and conventional cosmetic compositions. However, surprisingly, when combined with a silicone-aliphatic hydrocarbon wax (the alkyl silicone wax component of the examples) and an MQ resin and/or MT resin, this wax-resin combination minimizes color transfer of a cosmetic composition that is applied to a surface when that surface is contacted by a second surface.

(2) Moisture Barrier (Occlusivity) Property

In addition to providing excellent non-transfer benefits, the combination of a silicone-aliphatic hydrocarbon wax with either or both a fluorinated MQ silicone resin and an MT silicone resin can provide an effective barrier. This combination of silicone wax and silicone resin(s) serves to maintain skin in a moisturized state. When skin is more moisturized following application of a cosmetic composition this is perceived by the wearer as improved skin feel, or a greater level of comfort, compared to those cases where a greater amount of water is lost from the skin surface.

The ability of liquid lip compositions to provide a barrier against moisture loss from skin was assessed using Vitro Skin as a substrate. The Vitro Skin was conditioned by suspension above a glycerol/water solution in a sealed chamber. The section of skin was then placed on a film of petroleum jelly that had been applied to a glass microscope slide. A film of the liquid lip composition was then applied to the upper surface of the Vitro Skin and the rate of water loss (TEWL) from the skin measured with a TEWAMETER ex Courage-Khazakha. Lower TEWL values are observed for cosmetic films that are more effective in reducing the rate of water loss from the skin surface.

TABLE 3
Transepidermal Water Loss Measurements for Liquid Lip
Compositions Applied to Vitro Skin

| Example | Water Loss [units?] |
|---|---|
| Negative Control | 20 |
| (no applied film) | |
| Positive Control | 6.5 |
| (petroleum jelly film) | |
| Comp. Ex. 2 | 15.5 |
| Ex. 1 | 10.5 |
| Ex. 2 | 12.5 |
| Ex. 5 | 12.5 |

The cosmetic compositions of the invention (Examples 1, 2 and 5 and Comparative Example 2) provide a significant reduction in the rate of water loss from a Vitro Skin substrate and provide an effective barrier to water loss. However, the compositions of Examples 1, 2 and 5 achieve this highly desirable property without the presence of the dimethicone gum component of Comparative Example 2.

While the invention has been described in detail in connection with specific embodiments thereof, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Accordingly, the invention is not limited by the foregoing description.

1. A transfer resistant cosmetic composition with improved feel is provided which comprises:
a) a film-forming effective amount of at least one silicone-aliphatic hydrocarbon wax in which the aliphatic hydrocarbon substituent possesses from 24 to 60 carbon atoms;
b) a transfer resistant effective amount of at least one silicone resin selected from the group consisting of MQ resin and MT resin; and,
c) at least one volatile carrier,
the cosmetic composition being substantially free of dimethicone gum.

2. The cosmetic composition of claim 1 wherein the silicone wax is a silicone-aliphatic hydrocarbon wax in which the aliphatic hydrocarbon substitutuent possesses from 30 to 45 carbon atoms.

3. The cosmetic composition of claim 1 wherein the MQ resin is selected from the group consisting of non-fluorinated MQ resin, fluorinated MQ resin and mixtures thereof.

4. The cosmetic composition of claim 3 wherein in the non-fluorinated MQ resin, M is a unit of the general formula R′₃SiO_{1/2} and Q is a unit of the general formula SiO_{4/2}, the MQ resin optionally containing small amounts of D and/or T units, R′ being a hydrocarbon radical of from 1 to 6 carbon atoms.

5. The cosmetic composition of claim 4 wherein R′ is methyl.

6. The cosmetic composition of claim 3 wherein in the fluorinated MQ resin, M is a unit of the general formula R¹₃SiO_{1/2}, Q is a unit of the general formula SiO_{4/2}, the fluorine-containing group(s) are bonded to silicon atom(s) of M group(s) and the resin optionally contains up to 5 mole percent of D and/or T units, R¹ being a hydrocarbyl radical of from 1 to 6 carbon atoms.

7. The cosmetic composition of claim 6 wherein R¹ is methyl.

8. The cosmetic composition of claim 6 wherein the fluorine-containing group(s) are selected from the group consisting of those of the general formula: 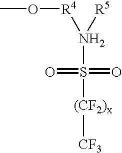
and those of the general formula:
—O—R⁴—(CF₂)ₓCF₃
wherein R⁴ and R⁶ each independently is an alkylene group possessing from 2 to 6 carbon atoms, 0≦x≦12 and each R⁵ independently is an alkyl group possessing from 1 to 6 carbon atoms.

9. The cosmetic composition of claim 1 wherein in the MT resin, M possesses the formula R₃SiO_{0.5}, R⁶RRSiO_{0.5}, R⁶R⁷RSiO_{0.5} or R⁶R⁷R⁸SiO_{0.5} wherein R is methyl and R⁶, R⁷ and R⁸ each independently is alkyl of from 1 to 6 carbon atoms, phenyl, diisostearoyl trimethylolpropane or dilauryltrimethylolpropane and T is a trifunctional siloxy unit of the formula R⁹SiO_{1.5} wherein R⁹ is alkyl of up to 20 carbon atoms optionally substituted with one or more hydroxyl groups and/or halogen atoms.

10. The cosmetic composition of claim 9 wherein M possesses the formula R₃SiO_{0.5} in which R is methyl and T possesses the formula RSiO_{1.5} in which R is methyl.

11. The cosmetic composition of claim 7 wherein the MT resin is a polymethylsilesquioxane.

12. The cosmetic composition of claim 1 wherein the volatile carrier is at least one member of the group consisting of volatile hydrocarbon oil and volatile silicone fluid.

13. The cosmetic composition of claim 12 wherein the volatile hydrocarbon oil is an isoparaffin of from 8 to 20 carbon atoms.

14. The cosmetic composition of claim 13 wherein the isoparaffin is at least one member selected from the group consisting of isododecane, isohexadecane, isoeicosane, 2,2,4-trimethylpentane and 2,3-dimethylhexane.

15. The cosmetic composition of claim 12 wherein the volatile silicone fluid is a cyclomethecone.

16. The cosmetic composition of claim 1 wherein silicone-aliphatic hydrocarbon wax (a) is present therein at a level of from about 3 to about 30 percent by weight, silicone resin (b) is present therein at a level of from about 1 to about 50 percent by weight and volatile carrier (c) is present therein at a level of from about 10 to about 90 percent by weight.

17. The cosmetic composition of claim 1 wherein silicone wax (a) is present therein at a level of from about 5 to about 20 percent by weight, silicone resin (b) is present therein at a level of from about 2 to about 30 percent by weight and volatile carrier (b) is present therein at a level of from about 15 to about 80 percent by weight.

18. The cosmetic composition of claim 1 wherein silicone wax (a) is present therein at a level of from about 8 to about 15 percent by weight, silicone resin (b) is present therein at a level of from about 5 to about 20 percent by weight and volatile carrier (b) is present therein at a level of from about 20 to about 70 percent by weight.

19. The cosmetic composition of claim 1, further comprising at least one additional component selected from the group consisting of pigments, colors, other waxes, emulsifiers, skin care ingredients, emollients and moisturizers.

20. The cosmetic composition of claim 1 which is a lipstick, lip gloss, makeup, mascara, eye liner, blusher, eye shadow or foundation.

21. The cosmetic composition of claim 19 which is a lipstick, lip gloss, makeup, mascara, eye liner, blusher, eye shadow or foundation.
